(19) United States
(12) Patent Application Publication
(10) Pub. No.: US 2010/0120044 A1
(43) Pub. Date: May 13, 2010

(54) MODIFIED POLYNUCLEOTIDES COMPRISING RIBOSE RINGS

(75) Inventors: Singer Thorsten, Solingen (DE); Löffert Dirk, Dusseldorf (DE)

Correspondence Address: Baker Donelson Bearman, Caldwell & Berkowitz, PC, 555 Eleventh Street, NW, Sixth Floor, Washington, DC, 20004, US

(73) Assignee: Qiagen GmbH, Hilden (DE)

(21) Appl. No.: 12/593,740

(22) PCT Filed: Mar. 31, 2008
(86) PCT No.: PCT/EP08/02550

§ 371 Date: Nov. 3, 2009

Related U.S. Application Data
(60) Provisional application No. 60/909,065, filed on Mar. 30, 2007.

(51) Int. Cl. C12Q 1/68; G01N 33/53; C07H 21/00; C07H 1/06

(52) U.S. Cl. 435/6; 436/94; 536/25.3; 536/25.4

(57) ABSTRACT

The invention pertains to different methods employing the use of a polynucleotide comprising ribose rings which carry a modification at the 2′-OH group.

The present invention pertains to a method for performing a detection or polymerization reaction comprising the use of a modified polynucleotide comprising ribose rings. The invention further pertains to a method for isolating, purifying or preparing a target nucleic acid from a mixture of at least two different types of nucleic acids. The modified polynucleotides used according to the present invention comprise ribose rings that are covalently modified at the 2′-OH position. Further a method is provided for obtaining a polymerization agent which efficiently utilizes respectively modified polyribonucleotides.

RNA serves as an essential component of every modern biological study. It provides a raw material for medical diagnostics, drug design, recombinant protein production, bioinformatics and almost every area concerning the pharmaceutical and biotechnology industries.

RNA is an essential and universal component of all organisms. There are three major types of RNA; these are messenger RNA (mRNA), transfer RNA (tRNA) and ribosomal RNA (rRNA), the latter being the most common type. Furthermore, in the recent years several types of small RNAs were discovered such as siRNA and miRNA. In addition, some viruses encode their genes in the form of RNA such as the retroviruses, HIV being one example of this type. Other RNA forms include small infective RNA loops called viroids, PSTV being one example of this type. RNA has many diverse functions such as in the production of proteins and the storage of genetic information.

All RNA molecules are linear macromolecules composed of repeated monomers (ribonucleotides) comprising a base, a ribose sugar and a phosphate. There are four principal bases: uracil, cytosine, guanine and adenine; the order in which they are connected together, the sequence, leads to many of the unique properties of RNA.

RNA differs chemically from DNA in two major ways. Firstly, it contains uracil instead of thymine, and secondly, RNA has a 2′-OH group on the ribose sugar instead of 2′-H found on the deoxyribose sugar of DNA. Natural RNA has the 2′ carbon atom bonded to two other carbon atoms (C1′ and C3′), a hydrogen atom and an oxygen atom that forms part of a hydroxyl group (here called the 2′-OH group). The 2′-OH group endows RNA with many of its unique properties such as structure, reactivity and instability. The 2′-OH group can also assist in the cleavage of the phosphodiester bonds between ribonucleotides leading to chain cleavage and hence RNA degradation.

When RNA is manipulated for any number of common laboratory practices, its inherent instability leads to considerable technical and experimental difficulties. For example, measuring the abundance and size of a particular mRNA species is frequently considered essential to understanding the function of a gene. When the particular mRNA under study is degraded, even to a small extent, such measurements become impossible to carry out reliably or accurately. Another example would be the synthesis of a cDNA copy of a mRNA, where degradation of the mRNA precludes any possibility of obtaining a full and representative cDNA. Such cDNA copies are considered essential experimental tools because they allow a full and accurate characterization of the gene such as its pattern of expression and chromosomal location. Furthermore the cDNA is essential to produce recombinant protein.

Protecting RNA from degradation whilst maintaining its biological activity is an essential task for any researcher or technician. However, the difficulty of removing nuclease activity from the RNA and the ease of accidentally introducing it, often precludes successful RNA manipulation to all except the most experienced. The cost and time considerations of RNA shipping and storage, equipment sterilisation, purchase of disposable plastic ware, training personnel and repeating failed experiments are a significant part of any laboratory budget.

The most important aspect of purifying RNA is to prevent degradation by RNases. RNases can be introduced e.g. from three sources: (1) intra-cellular sources due to carry-over from the experimental sample, (2) from external sources such as the researcher's skin secretions and (3) purified RNase used for DNA purification. RNases are truly ubiquitous; they can be found in finger tip secretions, dust, microbes, nearly all biological materials and even slight contamination will inevitably lead to RNA degradation. Compounding the problem is the common use of highly concentrated RNase in many DNA purification kits.

There are two principal means by which the 2′-OH group of ribonucleotides can be modified (a) enzymatically and (b) chemically. Enzymatic modification of the 2′-OH group arises from highly specific enzyme-catalysed reactions. For example, ribonucleotide reductase modifies the monomer ribonucleoside diphosphate, whereas an entire RNA molecule will not be recognised as a substrate. Another example is the methyl transferases that use an entire RNA molecule as a substrate but modify only a few 2′-OH groups per molecule.

The chemical synthesis of RNA and DNA is well known and many companies provide custom RNA and DNA synthesis (for review, see Eaton, (1995) Annu. Rev. Biochem. 64, 837). A considerable body of published work exists describing the different approaches to its synthesis (for review, see: Usman and Cedergreen (1992) TIBS 17:334). Protective groups have been reviewed (Greene and Wuts (1991) Protective Groups in Organic Synthesis, 2.sup.nd Ed. Wiley Interscience). The most prominent route for preparation of 2′-modified ribopyrimidines is through the introduction of nucleophiles to the corresponding 2,2′-anhydropyrimidine precursor. This reaction is limited to preparation of 2′-halides, 2′-azide, 2′-thiolates (Moffatt, (1979) In: Nucleoside Analogues, Ed. Walker, pp. 71-163, NY, Plenum., Townsend, (1988) Chemistry of Nucleosides and Nucleotides, pp. 59-67, NY, Plenum), 2′-azido (Verheyden, et al., (1971) J. Org. Chem. 36:250) and 2′-amino ribonucleoside (Wagner, et al., (1972) J. Org. Chem. 37:1876). Methylation of the 3′,5′-protected precursor gives 2′-O-methyl ribonucleosides (Sproat, et al., (1991) Oligonucleotides and Analogues: A Practical Approach, ed. F. Eckstein, pp. 49-86, NY. Oxford Univ. Press), and similarly 2′-O-alkyl and 2′-O-allyl derivatives have been made (Sproat, (1991) Nucleic Acids Res. 19:733, Lesnik, et al., (1993) Biochemistry. 32, 7832). Other modifications include 2′-methyl (Matsuda, et al., (1991) J. Med. Chem. 34:234), 2′-phenyl, 2′-alkyl ribonucleosides (Schmit (1994) Synlett. 234), 2′-acetylated (Imazawa, et al., (1979) J. Org. Chem. 44:2039), 2′-fluoro, 2′-trifluoromethyl (Schmit, (1994) Synlett. 241), 2′-mercapto (Imazawa, et al., (1975) Chem. Pharm. Bull. 23:604) and 2′-thio ribonucleosides (Divakar, et al., (1990) J. Chem. Soc. Perkin Trans. 1:969). 2′-Fluoro, 2′-O-methyl, 2′-O-propyl and 2′-O-pentyl nucleotides have each been incorporated into oligoribonucleotides (Cummins, (1995) Nucleic Acid Res. 23:2019). In each case the substrates and products are non-polymerised, that is they exist as simple monomers and not in the polyribonucleotide (RNA) form.

Several other approaches and background information on RNA modifications and the technology to obtain modified RNA are described in U.S. Pat. No. 6,867,290, herein fully incorporated by reference.

However, even though RNA modifications may protect RNA from degradation, said modifications may lead to problems when a polymerization/amplification of said ribonucleotide is intended in particular, if the RNA is reversed transcribed. This as the modification of the 2′-OH group changes the structure of the RNA. This is especially the case, when more than 25% of the ribose rings of the poly-ribonucleotide is modified. Of course, the effect on the structure of the poly-ribonucleotide depends on the type of modification chosen. However, as soon as higher modification percentages are used (in particular more than 25%, more than 50%, more than 75% and more than 90%) this problem may arise.

Hence, even though attaching modifications to the 2′-OH group leads to an efficient stabilization, the further processing of the poly-ribonucleotide is often hindered. For this reason it is very often necessary, to eliminate the modification prior to further processing the poly-nucleotide. This often requires purification and cleaning steps. This reduces the yield and furthermore has the disadvantage, that it requires time and is therefore also cost intensive.

It is thus an object of the present invention, to provide an efficient method for polymerizing/amplifying a modified poly-ribonucleotide.

Furthermore, it is an object of the present invention to provide methods for allowing an easy processing of modified polynucleotides comprising ribose rings and also to provide an efficient purification/isolation method for target nucleic acids.

According to a first aspect of the present invention a method for performing a detection or polymerization reaction is provided, comprising the following steps:
using a modified polynucleotide comprising ribose rings, wherein at least part of said ribose rings comprise a modification at the 2′-OH position, wherein said modification enables immobilisation of said modified polynucleotide to a support material
immobilising said modified polynucleotide to a support material
performing the detection or polymerisation reaction using the polynucleotide as a template or target for detection.

The detection or polymerization method according to the present invention utilizes the use of a modified polynucleotide, comprising ribose rings, which is immobilized via its modification to a support material. The binding of the modified polynucleotide to the surface of a respective solid support alleviates the further processing of said polynucleotide. It was surprisingly found that immobilization of the modified polynucleotide to the support via the modification is sufficient in order to securely anchor the polynucleotide comprising ribose rings to the support. Thus, handing and pipetting steps can be reduced, as the processing steps can be performed e.g. in a single vessel/receptacle (as support material). Usually, modified polynucleotides are bound/immobilized to respective support materials by hydrophobic interactions. The detection and/or polymerization reaction may also be carried out while the modified polynucleotide is still bound to said support.

The support material may be for example a solid support such as a microtiter plate, PCR reaction tubes or membranes or particles. Respective supports are well known to the skilled person and may also include but are not limited to beads, magnetic particles, columns, membranes or filters. They may include a mineral or a polymer.

The polynucleotide comprising ribose rings is preferably a polyribonucleotide. It is preferably a RNA and may be mRNA, tRNA, rRNA, viral RNA, small interfering RNA such as miRNA or siRNA, synthetic RNA such as chemically synthesised or in vitro transcribed forms, or any other form of RNA, such as hnRNA and viroid RNA. The RNA may be a mixture of different types of RNA and may be in single- or double-stranded form, linear or circular and even contain internal regions of secondary structure such as is commonly found in tRNA, mRNA and viral RNA. According to the present invention a polynucleotide generally has a sequence length of more than about 15, 50, 70, 80 and preferably more than about 100 bases. A preferred length for a polynucleotide is at least 1000 bases. The mRNA may or may not have a cap and/or polyA tail. The mRNA, rRNA, or viral RNA used in the present invention is preferably naturally-occurring. A naturally-occurring RNA according to the present invention typically comprises a nucleotide sequence which is found in nature and which generally encodes a polypeptide having biological activity, or such a nucleotide sequence which is modified, for example to alter in some way the biological activity of the polypeptide encoded thereby. Whilst the naturally-occurring RNA is preferably obtained by transcription from a suitable template, itself usually naturally-occurring, in some cases the naturally-occurring RNA can be obtained synthetically. mRNA according to the present invention does not encompass simple homopolynucleotides (polyA, polyU, polyG and polyC) which can be generated synthetically but are biologically non-functional.

In the polymerization reaction the polynucleotide is used as a template in order to produce a second complementary strand of RNA or DNA. The production of double stranded nucleic acid based on the template of modified polynucleotides, enables and alleviates the further processing of the original polynucleotide. According to one embodiment, the modified polynucleotide is polymerised by the use of a polymerase. Accordingly, the percentage of modification of the ribose rings may be chosen such that a polymerisation occurs when using said modified polynucleotide is used as a template. Furthermore, also specially adapted enzymes may be used. Also the kind of modification may process the polymerization and can thus be adapted.

According to a further embodiment, it is possible to remove the modification prior to the polymerization reaction but after the modified polynucleotide was processed (e.g. isolated/purified from a sample). This allows the use of conventional polymerases and allows the amplification with a high yield. E.g. in case a reversible modification is used, the modification is removed prior to the amplification reaction. One suitable example is the use of photoreactive modifications, wherein the modification is removed e.g. upon irradiation, thereby releasing the polynucleotide.

Modification at the 2′-OH position is preferably substantially regiospecific. Thus, there is preferably substantially no modification of the bases, phosphodiester bonds and/or any other position within the ribose ring and hence the RNA chain other than the 5′-OH and 3′-OH groups. In this way, the polynucleotide retains important properties of the RNA. For example, advantageously, the polynucleotide is preferably modified so that a single strand of the polynucleotide is replicable by a nucleic acid polymerase to generate a second strand of polynucleotide complementary to the single strand. This can also be achieved by using reversible modifications.

The extent of the modification of the 2′-OH position of the ribose rings may vary according to the intended further processing of the modified polynucleotide. Generally, it is intended that the polynucleotide comprising ribose rings is modified such that a proportion of the ribose rings is modified at the 2′-OH position. The modification used is preferably sufficient in order to protect the polynucleotide against nuclease degradation, especially against cellular endonucleases and/or intracellular concentrations of nucleases.

According to a further embodiment, the ribose rings of said polynucleotide are modified with at least two different types of modifications. This embodiment has the advantage, that for example different modifications may be used for immobilizing said modified polynucleotide to the solid support and for protecting said polynucleotide form degradation. This, as it has been shown, that certain modification of the polynucleotide, even though suitable for immobilization of the polynucleotide to the solid support, hinder the further processing of the modified polynucleotide by a polymerization agent. This, as such modifications often alter the secondary structure such, that the template is not longer recognized by the polymerization agent. Hence, it is beneficial to use one type of modification for the attachment to the solid support and another type of modification for additionally protecting said polynucleotide from degradation. One example for a respective embodiment would be the use of long carbon chains (for example C8 to C18) for immobilizing the polynucleotide to the solid support. As long carbon chains may alter the secondary structure in case they are used in a too high percentage for modification, it is preferred that a modification percentage is used, that allows an efficient immobilization of the polynucleotide to the solid support. Further modifications may then for example be performed by using the formyl group, which also protects the polynucleotide from degradation but do not substantially hinder the polymerization reaction. As is outlined above, also reversible modifications can be used in order to simplify processing by polymerases.

Hence, by choosing specific modifications in a balanced proportion, optimized conditions for protecting and immobilizing said modified polynucleotide may be achieved.

The percentage of the overall modification is preferably less than 75%, less than 60%, less than 50% less than 40%, less that 30% and preferably even less than 25%.

Suitable methods for measuring the percentage modification of the polynucleotide comprising ribose rings are described in U.S. Pat. No. 6,867,290, herein incorporated by reference.

Regiospecificity of the reaction can be determined by subjecting an identical sequence of DNA (or preferably single stranded DNA bearing uracil as a replacement to thymine), to identical reaction conditions as used for RNA. It is expected that the DNA is not substantially modified as measured by incorporation of radioactivity, gel electrophoresis mobility, mass spectrometry, HPLC or any other analytical means used if the reaction is regiospecific for the 2′-OH group.

The modification at the 2′-OH position may be such that the entire OH of the 2′C of the ribose ring is replaced by a reactant group R as in 2′-R or by OR having 2′-OR where the —O— group may or may not originate from the 2′-OH group. Accordingly, the substituent at the 2′-OH position in this case is R or OR respectively. One aim of the modification is to protect the molecule to a significant extent from degradation. Degradation may be a result of nucleases, metal ions and/or high temperatures, high pH or other chemical or physical conditions.

Preferably, said modification of the ribose rings is covalent. Regarding certain embodiments it is advantageous that the modification is reversible. For example, modifications may be chosen, where the modification may be altered by the addition of certain agents, for example salts. Another suitable example of a reversible modification is the use of photoreactive modifications, such as with 4,5-dimethoxy-2ribobenzyl chloroformate. Thereby a light-sensitive side chain is introduced allowing easy removal of the modifying gramp by irradiations at a suitable wave length. Also other photoreactive gramps are generally known and can be used according to the teachings of the present invention. Generally, for certain embodiments it may be sufficient, that the modification is stable over several days (for example 3 to 10 days) as the material would not be used for a longer time as it is e.g. used up.

According to one embodiment, said covalent modification is selected from the group consisting of:
modifications altering the overall charge of said polynucleotide,
modifications providing an affinity tag to said polynucleotide,
modifications altering the hydrophobicity of said polynucleotide,
modifications altering the affinity to thiophillic matrices
reversible modifications introducing a light-sensitive side chain.

According to one embodiment, said covalent modification of the ribose rings is a modification which alters the overall charge of said polyribonucleotide. Polyacrylic acid or poly-aspartatic acid may be used in order to enhance the overall charge of the polynucleotide. Polyethylene imine, polyvinylamine or poly-lysine may be used in order to reverse the overall charge of the RNA.

Carbon chains, in particular long carbon chains such as C2 to C25 or C6 to C25 (preferably C8 to C18) or perfluorinated carbon chains may be used in order to change the hydrophobicity of the polynucleotide. Long carbon chains are also especially suitable to immobilize the modified polynucleotide on a support such as a microtiter plate. However, also other hydrophobic substituents may be used for modification.

Said modification may also comprise a substituent OR or OR′, wherein R is selected from alkyl, alkenyl, alkynyl, haloalkyl, aminoalkyl, haloalkoxyalkyl, aminoalkoxyalkyl, aryl, alkylaryl, arylalkyl, arylalkenyl, alkanoyl, alkenoyl, haloalkanoyl, dihaloalkanoyl, trihaloalkanoyl, haloformylalkanoyl, aminoalkanoyl, arylalkanoyl, arylalkenoyl, alkoxyalkanoyl, aryloxyalkanoyl, alkylarylalkanoyl, azidoalkanoyl, carboxyalkanoyl, carboxyalkenoyl, carboxyalkynoyl, haloarylalkanoyl, aminoarylalkanoyl, alkylaminoarylalkanoyl, haloalkenoyl, haloalkynoyl, alkylsilanyl, trialkylsilanyl alkoxycarbonyl, alkylthioalkoxyalkoxycarbonyl, alkenyloxycarbonyl, alkoxyalkoxyalkyl, alkoxyalkyl, alkylthioalkyl, alkylsulfonyl, diarylphosphone, wherein the aforementioned substituents may be optionally substituted; or a substituent R′, wherein R′ is selected from alkyl, alkenyl, alkynyl, haloalkyl, aminoalkyl, halo, amino, alkylamino, aryl, alkylaryl, arylalkyl, wherein the aforementioned substituents may be optionally substituted.

Furthermore, said modification may also comprise a substituent OR or OR′, wherein R is selected from C1-C10 alkyl, C1-C10 alkenyl, C1-C10 alkynyl, C1-C10 haloalkyl, C1-C10 aminoalkyl, C1-C10 haloalkoxyalkyl, C1-C10 aminoalkoxyalkyl, C6-C14 aryl, C6-C14 alkylaryl, C6-C14 arylalkyl, C6-C14 arylalkenyl, C1-C10 alkanoyl, C1-C10 alkenoyl, C1-C10 haloalkanoyl, C1-C10 dihaloalkanoyl, C1-C10 trihaloalkanoyl, C2-C10 haloformylalkanoyl, C1-C10 aminoalkanoyl, C6-C14 arylalkanoyl, C6-C14 arylalkenoyl, C1-C10 alkoxyalkanoyl, C6-C14 aryloxyalkanoyl, C6-C14 alkylarylalkanoyl, C1-C10 azidoalkanoyl, C1-C10 carboxyalkanoyl, C1-C10 carboxyalkenoyl, C1-C10 carboxyalkynoyl, C6-C14 haloarylalkanoyl, C6-C14 aminoarylalkanoyl, C7-C15 alkylaminoarylalkanoyl, C1-C10 haloalkenoyl, C1-C10 haloalkynoyl, C1-C10 alkylsilanyl, C3-C10 trialkylsilanyl C1-C10 alkoxycarbonyl, C3-C18 alkylthioalkoxyalkoxycarbonyl, C1-C10 alkenyloxycarbonyl, C3-C18 alkoxyalkoxyalkyl, C2-C12 alkoxyalkyl, C2-C12 alkylthioalkyl, C1-C10 alkylsulfonyl, C12-C28 diarylphosphone, wherein the above mentioned substituents may be optionally substituted; or a substituent R′, wherein R′ is selected from C1-C10 alkyl, C1-C10 alkenyl, C1-C10 alkynyl, C1-C10 haloalkyl, C1-C10 aminoalkyl, halo, amino, C1-C10 alkylamino, C6-C14 aryl, C6-C14 alkylaryl, C6-C14 arylalkyl, wherein the above mentioned substituents may be optionally substituted.

Preferably, R and/or R′ is selected from methyl, ethyl, vinyl, allyl, ethynyl, 2-chloroethyl, 2-aminoethyl, ethyloxyethyl, methoxymethyl, methylthiomethyl, methoxyethoxymethyl, (2-chloroethyl)oxyethyl, (2-aminoethyl) oxyethyl, phenyl, 4-methylphenyl, benzyl, cinnamyl, formyl, acetyl, propanoyl, butanoyl, pentanoyl, hexanoyl, heptanoyl, octanoyl, nonanoyl, pivaloyl, isobutanoyl, isopentanoyl, carboxyacetyl, chloroformylnonanoyl, 3-carboxypropanoyl, 4-aminobutanoyl, 4-chlorobutanoyl chloroacetyl, dichloroacetyl, trifluoroacetyl, trichloroacetyl, 3-azidopropanoyl, 4-azidobutyryl acryloyl, propioloyl, crotonoyl, benzoyl, diphenylacetyl, phenoxyacetyl, methoxyacetyl, methoxycarbonyl, 2-(methylthiomethoxy)ethoxycarbonyl, vinyloxycarbonyl, 4-methylbenzoyl, 4-chlorobenzoyl, 2-methylaminobenzoyl, 2-aminobenzoyl, 4-aminobenzoyl, 4-nitrobenzoyl, cinnamoyl, silanyl, trimethylsilanyl, triethylsilanyl, tripropylsilanyl, triisopropylsilanyl, t-butyldimethylsilanyl, 2-chlorophenyl (4-nitrophenyl)phosphono, methylsulfonyl; and R′ is selected from methyl, ethyl, vinyl, allyl, ethynyl, t-butyl, 2-chloroethyl, 2-aminoethyl, ethyloxyethyl, phenyl, benzyl, fluoro, chloro, bromo, iodo, amino.

Said modification may also be selected from the group consisting of
polyacrylic acid or polyaspartatic acid
polyethylenimine, polyvinylamine or polylysine
C1 to C20 or C6 to C20, preferably C8 to C18 carbon chains or perfluorinated carbon chains
Biotin or poly-histidine
siloxane with a formulation of “—O—SiR₃”, where R can be an organic moiety with 1 to 6 carbons, a phenyl residue or a “—O—SiR₃” group with R=aliphatic group CₙH₂ₙ₊₁ with n=1-8 or aromatic group,
modifications comprising a label, which is preferably selected from the group consisting of a fluorescent label, a radioactive label, an enzyme, a ligand or an affinant for a label.

Further suitable modifications are described above in conjunction with the prior art discussed above and herein incorporated by reference. According to a further embodiment, the modification alters the affinity to thiophilic matrices. It is also feasible to attach an affinity tag as modification to said polynucleotide, such as for example biotin or polyhistidin. Respective modifications are especially suitable for detection uses/applications.

Further modifications that may be used according to the present invention and methods for attaching them to polynucleotides comprising ribose rings are described in U.S. Pat. No. 6,967,290, the teaching of which is herein fully incorporated by reference.

According to a further embodiment, said modification comprises a label, which is preferably selected form the group consisting of a fluorescent label, a radioactive label, an enzyme, a ligand or an affinent for a label.

As solid supports, particularly supports such as nucleic acid binding matrices, membranes, particles, laboratory equipment such as chips, microtiter plates, tubes or vessels can be used.

According to one aspect, the present invention provides the use of a polynucleotide in particular RNA, a proportion of the ribose rings of which are covalently modified at the 2′-OH position, as a probe. The probe may be labelled, for example, with a fluorescent or radioactive label. For example, modified mRNA may serve as a labelled probe for hybridisation, finding utility, for example, in “biochip” applications used to study gene expression. These are examples for detection methods.

Accordingly, said polymerisation reaction may comprise a polymerase reaction such as a reverse transcription, PCR or isothermal amplification using a polymerisation agent.

In a further aspect, the present invention provides a method for the replication of a polynucleotide comprising ribose rings, which comprises obtaining a polynucleotide comprising modified ribose rings as described above, and replicating the modified polynucleotide to form a complementary polynucleotide using a nucleic acid polymerase. Because modification of polyribonucleotide in accordance with the present invention can provide a replicable polynucleotide which is relatively stable to laboratory manipulation, the polynucleotide may be used in a range of applications as a substitute for DNA. The complementary polynucleotide may comprise an RNA, DNA or hybrid or modified forms thereof.

For example, the complementary polynucleotide may comprise a cDNA and the nucleic acid polymerase may comprise a DNA polymerase. Such polymerases are discussed in detail below.

The copying of mRNA into cDNA is an important method for obtaining fully representative copies for use in applications including cDNA cloning, DNA sequencing, protein production for drug screening programs and understanding the function of a particular gene. Conventionally, all require the activity of reverse transcriptase which is associated with many associated problems such as inhibition.

The synthesis and cloning of cDNA involves a complex series of enzymatic steps in order to copy the mRNA into double-stranded DNA and cloning this into a DNA vector. As used herein the term cDNA refers to a complementary DNA molecule synthesized using a ribonucleic acid strand (RNA) as a template. Many approaches are known for cDNA cloning, all have tried to preserve as much of the original sequence as possible (Okayama and Berg, (1982) Mol. Cell. Biol. 2:161, Gubler and Hoffman, (1983) Gene 25:283).

Conventionally, problems can occur at least at one or more of three stages, 1) mRNA isolation, 2) first strand cDNA synthesis or 3) second strand synthesis. When the mRNA starting material is degraded, incomplete forms of the cDNA are an inevitable result. One application of the present invention is to stabilise the mRNA molecule in order to isolate complete copies of the mRNA. mRNA modified in accordance with the present invention can be used as a template for reverse transcriptase.

Obtaining a full length cDNA is one of the most difficult yet important tasks when characterising a gene. Most commonly, cDNA libraries are produced by the complete conversion of a mRNA pool into a cDNA copy (Gubler and Hoffman (1983) Gene 25:263-269) however the most common outcome is to produce an incomplete representation of the starting mRNA.

Methods to isolate full length cDNA copies of mRNA include: RACE (rapid amplification of cDNA ends) first described in 1988 as a method to isolate full length cDNAs using PCR (Frohmann, et al., (1988) Proc. Natl. Acad. Sci. USA 85, 8998-9002). Related methods have been reviewed (Schaefer, (1995) Anal. Biochem. 227:255-273). Although these methods can be successful for retrieving the 5′ and 3′ ends of single cDNA molecules, it requires considerable skill and depends in large part on the abundance of the mRNA and can only be done one at a time.

The method for the replication of the polynucleotide, according to the present invention, may further comprise a step of ligating to a vector a single- or double-stranded polynucleotide comprising the polynucleotide and the complementary polynucleotide. In this way, molecular cloning procedures may be accomplished using modified polynucleotides according to the present invention.

For this embodiment of the present invention, it is particularly preferred that the RNA is modified by formylation. Formylated polyribonucleotides such as RNA serves as an excellent template for reverse transcriptases. However, the optimum reaction conditions differ from those used for RNA. The most important difference is the divalent metal cation present in the reaction. Although MULV will reverse transcribe formylated RNA in the presence of MgCl.sub.2 e.g. at either 2.5 or 5 mM final concentration, it is preferred that the metal ion is manganese. Manganese is known to alter the specificity of many DNA polymerases such that their template specificity is relaxed. For example, reverse transcriptases will readily copy DNA templates and DNA-dependent DNA polymerases can use RNA templates in the presence of manganese ions. This may explain the enhanced template activity of formylated RNA in the presence of manganese ions. The Mn concentration is not especially limited, but the most preferred (optimum) Mn concentration is 1.2-1.4 mM. The reaction is less effective (with little cDNA product detected) with buffers containing in excess of 3 mM or less than 0.1 mM manganese. Mixtures of the two types of metal ion may also be employed in the present invention, such as a mixture of 1 mM manganese and either 0.5 or 1 mM magnesium ions. For details please also see U.S. Pat. No. 6,867,290.

A final Tris-HCl buffer (pH 8.4 at 22.degree.C.) concentration of 200 mM yields more cDNA product than the 50 mM specified in the product protocol of Superscript II (Life Technologies, USA). Increasing the Tris-HCl concentration further to 350 mM slightly reduces the cDNA yield.

Enzymes which can be used successfully for polymerization/amplification include Superscript II (Life Technologies), MULV RNase H.sup.+ (Promega), MULV RNase H.sup.− (Promega), Expand (Roche Molecular Biochemicals) and HIV-1 reverse transcriptase (Amersham Pharmacia). A mixture of Superscript II and AMV (Invitrogen, USA) may also be used successfully.

Formylated BMV RNA can be reverse transcribed in the presence of DMSO (e.g. 10% DMSO) which is known to reduce nucleic acid secondary structure, or in a Tris-HCl buffer pH 7.5 (e.g. at 22.degree.C.) or in KCl (e.g. 150 mM).

In a further aspect, the present invention provides, a method for producing a double-stranded oligo- or polynucleotide from a template, which comprises contacting the template with a plurality of mononucleotides comprising UTP, dTTP and/or dUTP, ATP and/or dATP, GTP and/or dGTP, and CTP and/or dCTP, in the presence of a nucleic acid polymerase and optionally a template primer under conditions to polymerise the mononucleotides to form a nucleic acid strand complementary to the template, wherein the template comprises an polyribonucleotide, a proportion of the ribose rings of which polyribonucleotide are covalently modified at the 2′-OH position to bear a substituent which enables replication of the template by the nucleic acid polymerase.

It has been surprisingly found that when the ribose rings of the oligo- or polyribonucleotide are modified in accordance with the present invention, the oligo- or polyribonucleotide produced thereby is capable of acting as a template for one or more of a variety of nucleic acid polymerases. Nucleic acid polymerases within the scope of the present invention include DNA polymerases, RNA dependent polymerases and RNA dependent RNA polymerases.

Among the RNA-dependent DNA polymerases are Superscript™ II (MMLV reverse transcriptase RNase H—), MMLV reverse transcriptase, HIV reverse transcriptase, AMV reverse transcriptase, RAV-2 reverse transcriptase, human T-cell leukemia virus type I (HTLV-I) reverse transciptase, bovine leukemia virus (BLV), Rous Sarcoma virus (RSV), Tth DNA polymerase, Tfl DNA polymerase, Bst polymerase, Taq DNA polymerase, Thermoscript, C.therm polymerase, displaythermo-RT or Klenow DNA polymerase.

Among the DNA-dependent DNA polymerases are DNA polymerase I;—Klenow fragment; T4 DNA polymerase; T7 DNA polymerase; Taq DNA polymerase, Tli DNA polymerase, Pfu DNA polymerase; Vent™. DNA polymerase; Deep Vent™. DNA polymerase; Bst DNA polymerase; Tth, Pfu Turbo™, Pfu(exo-), Pwo, Pyra™, Tfu, Klen-Taq, Taq2000™, AmpliTaq Stoffel fragment, Sequenase™, Tma, Vent® (exo-), Deep Vent® (exo-) or a DNA polymerase purified from Thermosipho africanus, Thermotoga maritima, Desulfurococcus mobilis, Methanobacterium thermoautotrophicum, Methanothermus fervidus, Pyrococcus furious, Pyrodictium occultum, Sulfolobus acidocaldarius, S. solfataricus, Thermococcus litoralis or Thermoplasma acidophilum.

Among the RNA-dependent RNA polymerases are Q beta replicase, and those derived from E. coli phage f2, R17, MS-2 or .o slashed.6, or from a virus family selected from the bromoviridae, flaviviridae, picornaviridae, potyviridae, tobamovirus, tombusviridae, leviviruses, hepatitis C-like viruses, and picornaviruses or from polio virus, yellow fever virus, tobacco mosaic virus, brome mosaic virus, influenza virus, reovirus, myxovirus, rhabdovirus and paramyxovirus.

Nucleic acid polymerases may be classified into four overlapping groups. Classification is based on the type of template copied (RNA or DNA) and the type of complementary nucleic acid strand that is produced (RNA or DNA). Although in vivo, nucleic acid polymerases have discrete activities, in vitro specificity for the template and the substrate mononucleotides is less stringent. As one example, in vitro certain DNA dependent DNA polymerases such as Taq and Tth DNA polymerase can also behave as RNA dependent DNA polymerases. Specificity depends in part on the buffer conditions, presence of metal ions and the type of mononucleotide triphosphate present. Lastly, many mutant forms of polymerases are known (for one example see; Gao et al., (1997) Proc. Natl. Acad. Sci. (USA) 94:407) that are less specific with respect to the template strand copies and the type of complementary strand produced. Accordingly, some enzymes appear in more than one of the above lists.

In a further aspect, a reverse transcription may take place as an example for a polymerization reaction.

In a further aspect, said polynucleotide comprising modified ribose rings is amplified by the use of a polymerization agent. Suitable polymerases are known and described above.

In a further aspect, the present invention provides the use of a nucleic acid polymerase for the production of a nucleic strand complementary to a template for the nucleic acid polymerase, wherein the template comprises an oligo- or polynucleotide comprising an oligo- or polyribonucleotide, a proportion of the ribose rings of which oligo- or polyribonucleotide are covalently modified at the 2′-OH position to bear a modification which enables replication of the template by the nucleic acid polymerase. The nucleic acid polymerase may be any of those nucleic acid polymerases defined above.

In a further aspect, the present invention provides use of an oligo- or polynucleotide as a template for a nucleic acid polymerase, wherein a proportion of the ribose rings of which oligo- or polyribonucleotide are covalently modified at the 2′-OH position, to bear a modification which enables replication of the template by the nucleic acid polymerase.

Either of these uses relate to, for example, reverse transcription or use in a polymerase chain reaction, including RT-PCR.

One example of a detection reaction is a hybridization reaction. Hence, in a still further aspect, the present invention provides the use of a polynucleotide comprising mRNA or viral RNA, a proportion of the ribose rings of which are covalently modified at the 2′-OH position, in a hybridisation reaction. Furthermore, a fluorescent dye may be used as detection reagent.

In accordance with this aspect of the invention it has been found that RNA modified in accordance with the present invention it is still capable of hybridising with nucleic acid. Because modified RNA is more stable to degradation than its unmodified counterpart, problems of degradation of RNA during and before analysis are avoided. There is no longer any need for extreme measures to be used to prevent RNA degradation such as those involving the use of ultra-clean working environments, or expensive inhibitors of RNases.

Typically, the hybridisation reaction comprises a hybridisation between a probe and a template comprising the poly-nucleotide, which may comprise a mixture of oligo- and poly-nucleotides such as those involved in a gene expression analysis.

Alternatively, the hybridisation reaction may comprise a hybridisation between a template and a probe comprising the poly-nucleotide.

The probe or the template may be immobilised to a solid phase such as a hybridisation membrane, a bead, a particle, a slide, a sheet, a gel, a microtitre strip, tube, fibre or capillary. In this aspect it is also advantageous to use different modifications, in order to allow e.g. immobilization and a second and/or third modification which protects said polynucleotide but, however, does not alter the structure of the polynucleotide such that it would seriously hamper the hybridization reaction.

The solid phase may be made of substances such as nitrocellulose, agarose, acrylamide, cellulose, latex, nylon, polystyrene, polycarbonate, polypropylene, PVDF (polyvinylidene fluoride), polytetrafluoroethylene, a silica-based material, a glass, a metal alloy, gold, a magnetic material or a paramagnetic material. Other materials were described also above.

The hybridisation reaction may comprise a blotting process typically using any one of the above solid phases.

The probe or template may be attached to another molecule or group of molecules. It is frequently desired that the probe or the template is labelled with a label, which may be a fluorescent label, a radioactive label, and enzyme, a ligand or an affinant for such a label. Fluorescent labels for carbohydrate labelling are described in U.S. Pat. No. 6,048,707 herein incorporated by reference. The molecules or group of molecules may itself comprise the label in the sense that the group of molecules is capable of causing a detectable reaction or capable of binding a detectable entity. The molecule or group of molecules may comprise a peptide, a poly-peptide such as an antibody, an enzyme, an affinity partner such as protein A or streptavidin, a receptor protein, a ligand such as biotin, dinitrophenyl, digoxigenin or other hapten or lectin, or a label such as fluorescein, rhodamine, Texas red, cy-5, TAMRA or a pigmented chromogenic, chemiluminescent or coloured marker.

The probe may comprise a branched DNA (bDNA) probe.

In a further embodiment, the poly-nucleotide may be bound to a third molecule such as an antibody-alkaline phosphatase conjugate.

The poly-nucleotide may comprise an antisense agent for use in an antisense hybridisation reaction for example in vivo.

In accordance with a further use, the poly-nucleotide has a specific binding affinity to a ligand and the hybridisation reaction comprises a hybridisation between the polynucleotide and a target comprising the ligand.

According to a further aspect of the present invention, a method for isolating, purifying or preparing a target nucleic acid from a mixture of at least two different types of nucleic acids is provided, wherein one of said nucleic acids is a polynucleotide comprising ribose rings. Said method comprises the following steps:
contacting said polynucleotide comprising ribose rings with a reactant capable of modifying the 2′-OH position of the ribose rings of said polynucleotide
reacting said polynucleotide with said reactant to produce a modified polynucleotide wherein at least part of said ribose rings comprise a modification at the 2′-OH position, wherein said modification changes the chemical or physical properties of said polynucleotide
isolating the target nucleic acid.

The described method is useful for isolating for example DNA and/or RNA as target nucleic acid.

For example, when purifying DNA from a sample, RNA is basically a contamination and accordingly should not be present in the purified target nucleic acid. Therefore, the prior art uses for example RNAses in order to degrade the RNA. However, alternative and improved methods are desired. A respective alternative method is provided by the present invention. According to this principle, the polynucleotide comprising ribose rings (the RNA) is modified at the 2′H position. The respective modifications have the effect that the polynucleotide comprising ribose rings adsorb/bind stronger to the support, e.g. a nucleic acid binding matrix, thereby preventing elution from the nucleic acid binding matrix. This has the effect, that the polynucleotide comprising ribose rings retains bound to the nucleic acid binding matrix and is thus not eluted together with the target nucleic acid, here the DNA. Accordingly, when using respective protocol according to the present invention, basically no RNA contaminations are found in the eluate. For example, acetic anhydride can be used in order to modify the polynucleotide comprising ribose rings.

There are several options in order to achieve a respective modification of the polynucleotide comprising ribose rings. E.g. the modification of the polynucleotide comprising ribose rings can be performed by at least one of the following steps
adding a modifying reagent during lysis of the sample comprising the target nucleic acid
adding a modifying reagent when the polynucleotide comprising ribose rings is contacted with the nucleic acid binding matrix
adding a modifying reagent while the polynucleotide comprising ribose ring is adsorbed to the nucleic acid binding matrix.

For example, the modification agent can be added to the lysis buffer. This has the effect, that the polynucleotide comprising ribose rings is already modified before contacted with the nucleic acid binding matrix. Good results are achieved with a respective protocol. However, often different lysis buffers are used for the isolation of the target nucleic acid (DNA) form different tissues/samples. In that case, one may want to avoid adapting the corresponding lysis buffer. In that case, a different protocol may be used for the modification. The principle of the present invention also works, when the polynucleotide comprising ribose rings is already bound/adsorbed to the nucleic acid biding matrix. Thus, it is possible to add the modification agent either to the binding buffer or even after binding of the nucleic acids to the nucleic acid binding matrix occurs. The polynucleotide comprising ribose rings is also efficiently modified while bound to the nucleic acid binding matrix. Due to the modification, the interaction between the modified polynucleotide comprising ribose rings and the nucleic acid matrix is enhanced such, that basically no or at least reduced amounts of the polynucleotide comprising ribose rings is found in the eluate. Thus, the method according to the present invention allows the purification of highly pure DNA, having no or at least a reduced amount of RNA contaminations.

Suitable modifications that can be used according to the present invention were described above and can also be used in conjunction with the described method. Basically any nucleic acid binding matrix can be used as support according to the principles of the present invention. For example any solid phase known to be useful for nucleic acid binding can be used, such as silica, glass, zeolithe, aluminium oxide, titandioxide, ceramic or polymeric nucleic acid binding matrixes. Other suitable supports such as tubes, vessels, chips and microtiter plates were described above. Respective supports can also be provided with a nucleic acid binding surface of the above mentioned materials.

Preferably, in particular when using a nucleic acid binding matrix having a hydrophobic surface, it is preferred that the modification introduces hydrophobic groups as modifications, thereby enhancing the overall hydrophobicity of the RNA. Thereby, the RNA binds better to the nucleic acid binding matrix. In case a charged nucleic acid binding matrix is used, one may also use modifications, which introduce the corresponding charge in order to allow a tight interaction of the modified polynucleotide comprising ribose rings with the nucleic acid binding matrix via the charges. For example, amino acids can be used for example in addition to the hydrophobic groups. Thereby, also the specificity of the interaction can be controlled.

As is outlined above, the modified RNA remains bound to the nucleic acid binding matrix due to the modification. In case reversible modifications are used, one may also elute the bound RNA from the nucleic acid binding matrix by reversing the modification. For example, in case photoreactive groups are used, one could irradiate the nucleic acid binding matrix carrying the bound polynucleotide comprising ribose rings in order to remove/reverse the modification, thereby allowing the elution of the polynucleotide comprising ribose rings.

Thereby a method is provided, which allows the specific isolation of RNA and/or DNA.

Thus, when using respective reversible modifications, the method of the present invention is also suitable to isolate highly pure RNA from a sample comprising other nucleic acids such as DNA. Furthermore, the method is useful for obtaining highly pure DNA from a sample comprising polynucleotides comprising ribose rings.

According to one aspect, the target nucleic acid is said polynucleic acid comprising ribose rings. Hence, said method is suitable for obtaining purified RNA. The modification may be advantageously used in order to obtain a respectively purified RNA.

According to one embodiment, said modified polynucleic acid is bound respectively associated via its modification to a support material. The isolation occurs then via said support (for example by extracting the RNA which is bound to a nucleic acid binding matrix such as particles). Suitable materials for respective supports are described above.

Said modified polynucleotide may be polymerised/amplified after isolation/purification/preparation. According to one embodiment, said modified polynucleic acid comprising ribose rings may be treated with a polymerization agent. The modified polynucleotide comprising ribose rings may be used as a template in order to generate either a reverse transcribed molecule or in order to perform a polymerase chain reaction. Said polymerisation/amplification may occur while said modified nucleic acid is immobilised/bound to said solid support.

According to a further embodiment, the target nucleic acid to be isolated or purified is DNA. In this case, the purification is achieved by separating said modified polynucleotide from the target nucleic acid DNA by using again the modifications of the ribose rings. Hence, said modified polynucleic acid can be separated from the target nucleic acid by using said modification of the ribose ring.

Suitable modifications of the polynucleotide comprising modifications of the ribose ring are described above and also belong to the described isolation/purification/preparation method. This pertains to all aspects described above including, but not limited to the different modifications, modification combinations, modification percentages.

According to a further embodiment, a polymerization agent is provided, which has been mutagenized to efficiently utilize a modified polynucleotide, comprising ribose rings.

As outlined above, depending on the type and percentage of the modification, the modified polynucleotide may not be directly susceptible to a treatment with a polymerization agent, as said modified polynucleotide is often not recognized any longer as a suitable template. Hence, said modified polyribonucleotide templates are often not comparably well reverse transcribed compared with the unmodified template by an RNA-dependent DNA polymerase (such as for example RMV, HIV, MMLV, reverse transcriptase enzymes) or a DNA-dependent DNA polymerase such as Tth-DNA polymerase. Said polymerases are only named by example and should not limit the present scope of the present invention. Further suitable examples were described above and also are usable for the respective embodiment.

In order to overcome the reduced reverse transcription and polymerization efficiency of polymerases when employing a modified polyribonucleotide (such as RNA) template, the present invention seeks to overcome this limitation by mutagenesis of the respective polymerases in order to obtain optimized enzymes. Mutagenesis is aimed to allow the efficient recognition and binding of the modified RNA template with increased capability to incorporate complementary nucleotides into the nascent growing cDNA strand as compared to the wild-type polymerase.

Hence, a method is provided for selecting a polymerization agent having the ability to efficiently utilize a polynucleotide comprising ribose rings, wherein at least part of said ribose rings comprise a modification at the 2′-OH position, wherein said modification changes the chemical or physical properties of said polynucleotide. Said method comprises the steps of:
mutagenising said polymerisation agent
incubating the mutagenised polymerisation agent together with said modified polynucleotide
detecting the efficiency of said polymerisation reaction.

Based on the detected efficiency, one may select an appropriate polymerase. The polymerases may also be adapted/evolved to efficiently recognize different modifications as a template. For example, they may be adapted to utilize differently modified polynucleotides. Samples for suitable modifications are described above and can be used in the described assay in order to evolve a respective polymerization agent which is adapted to efficiently recognize a polynucleotide carrying said respective modification.

According to one embodiment, the modified polynucleotide to be tested in the above assay carries more that 25% of modifications at its ribose rings.

Preferably, the polymerization agent is a polymerase. It may be selected from the group consisting of RNA-depending polymerases, DNA-dependent DNA polymerases, DNA-dependent RNA polymerases (see above).

The reaction usually comprises also a reaction buffer containing optionally salts, dNTP's and one or more oligonucleotides if desired. Oligonucleotides are especially suitable in order to initiate the reaction.

The polymerization reaction may either be isothermal, or may be a polymerase chain reaction employing heating steps.

Suitable mutagenesis methods which may be used in order to obtain mutagenesed enzymes are well known in the state of the art (for example please refer to Woycechowsky, Vamvaca “Novel enzymes through design and evolution”; Kaur, Sharma “Direct evolution: an approach to engineer enzymes”; Soumillion, Fastrez “Novel concepts for selection of catalytic activity”; Chen “Enzyme engineering: rational redesign versus directed evolution”; Antikainen, Martin “Altering protein specificity: techniques and applications”.

The present invention is now described by way of non-limiting examples:

### EXAMPLE 1

Modification of RNA for irreversible binding of RNA on membranes as an example for a nucleic acid binding matrix to allow isolation of DNA (for example for plasmid preparation) without the necessity to use RNase:

2 μg of RNA isolated with a RNeasy Mini Kit (Qiagen, Hilden) according to the standard protocol was modified according to the STABMRT Mini Kit protocol (RNAworks, Montpellier) with different reagents leading to modified RNA with different carbon chain length modification at the 2′-OH-position of the riboses. Isolated RNA was used in order to use the most difficult experimental conditions.

Modified and unmodified RNA in water was applied to a spin column and centrifugated:

a) RNeasy Mini Spin column (glass fibre membrane; Qiagen, Hilden)
b) Spin column with PVDF membrane

The flow-through was analysed for the presence of RNA on an agarose gel. In case no RNA was detectable by agarose gel analysis, this proves that the respective RNA sample was retained by the membrane. Usually, under the non-chootropic buffer conditions of the experiment, the RNA would be expected to be contained in the flow-through.

RNA was either used in an intact form or partially degraded to simulate hydrolytic degradation during an—e.g. alkaline—lysis procedure for plasmid isolation (intact vs. degraded)

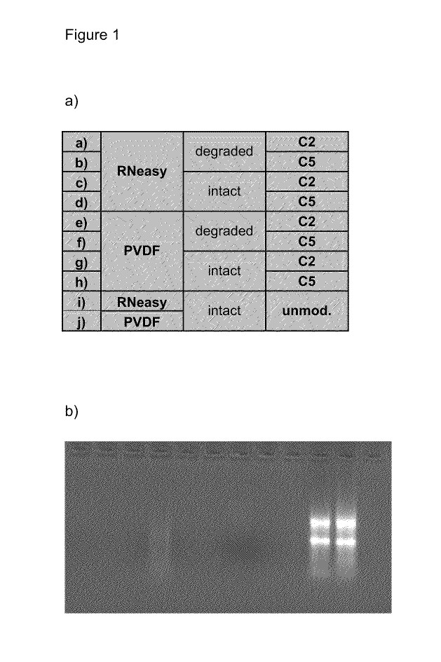

15 μl each of the flow-through were analyzed on a 1% formaldehyde agarose-gel. Intact unmodified RNA was used as reference sample. FIGS. 1a and 1b outline the experimental setting and the results. The abbreviations have the following meaning:

“C2” acetyl modification
“C5” valeryl modification
PVDF polyvinylidenefluoride

The results demonstrate that whereas the unmodified RNA is completely contained in the flow-through, modified RNA regardless it's quality (intact or degraded) is retained by the spin columns. This proves that the performed modification of RNA, e.g. during a plasmid (or other DNA) isolation procedure followed by binding to an appropriate nucleic acid matrix such as a membrane can efficiently remove RNA from a samples without the need to use RNases and this being independent of the size or quality of the RNA.

There is a broad series of other commercially available substances with different properties which can be used to modify RNA according to this protocol to design the physical and/or chemical properties according to the intended downstream application. E.g. amino acid anhydrides may provide a positively charged amino function (pH<7) or 4,5-dimethoxy-2-nitorbenzyl chloroformate can be used to introduce a light-sensitive side chain allowing easy removal by irradiation at a suitable wavelength.

### EXAMPLE 2

### RNA Modification Protocols

Subsequently, different RNA modification protocols are described that can be used in conjunction with the present invention.

a. Standard Modification Protocol

A respective protocol is in particular useful in case the RNA is present in high amounts.
1) Pipet 4 μl pf pre-cooled (4° C.) catalyst (1-methylimmidazole) into a microcentrifuge tube
2) Add up to 5 μg of RNA in a total of 4 μl of water, mix and place on ice
3) Add 40 μl of pre-cooled (4° C.) reaction solution (e.g. acetic anhydride in case of “C2”) and mix by pipetting
4) Incubate 5 minutes on ice
5) Add 150 μl of 96-100% ethanol and 5 μl of 3M NaCl, place in a freezer for 5 minutes
6) Apply to RNeasy Mini Spin column (Qiagen, Hilden, Germany) and centrifuge for 60 sec at >15.000×g; discard flow-through
7) Apply 500 μl Buffer RPE (Qiagen, Hilden, Germany) and centrifuge for 15 sec at >15.000×g; discard flow-through
8) Repeat step 9 and centrifuge an additional 60 sec at >15.000×g
9) Transfer column to a fresh tube, add 50 μl of water or Buffer EB (Qiagen, Hilden, Germany) and centrifuge for 60 sec at >15.000×g
10) Apply another 50 μl of water or Buffer EB and centrifuge again for 60 sec at >15.000×g

The eluate contains the modified RNA.

b.) Direct Lysis Protocol

The respective protocol is useful when the RNA is present in a sample which needs to be lysed for extracting the target nucleic acid.
1) Weigh 5-25 mg of tissue sample and add 100 μl of catalyst (1-methylimmidazole)
2) Grind and lyse your tissue using a polytron or piston
3) Add 400 μl of pre-cooled (4° C.) reaction solution (e.g. acetic anhydride in case of “C2”) and briefly vortex
4) Incubate 10 minutes at room temperature
5) Add 400 μl of Solution L (RNAworks, Montpellier, France)
6) Centrifuge 2 minutes at >15.000×g
7) Transfer supernatant into a new microcentrifuge tube and add 900 μl of 96-100% ethanol
8) Apply to RNeasy Mini Spin column (Qiagen, Hilden, Germany) and centrifuge for 60 sec at >15.000×g; discard flow-through
9) Apply 500 μl Buffer RPE (Qiagen, Hilden, Germany) and centrifuge for 15 sec at >15.000×g; discard flow-through
10) Repeat step 9 and centrifuge an additional 60 sec at >15.000×g
11) Transfer column to a fresh tube, add 50 μl of water or Buffer EB (Qiagen, Hilden, Germany) and centrifuge for 60 sec at >15.000×g
12) Apply another 50 μl of water or Buffer EB and centrifuge again for 60 sec at >15.000×g

The eluate contains the modified RNA.

c.) On-Membrane Modification Protocol

This protocol is useful when the RNA is modified while bound to the nucleic acid binding matrix.

Follow your/a standard nucleic-acid purification protocol until RNA is bound on the membrane (before washing steps).
1) Pipet 20 μl of catalyst (1-methylimmidazole) into a 1.5 ml tube, add 80 μl of reaction solution (e.g. acetic anhydride in case of “C2”), mix by pipetting and immediately apply onto the centre of the membrane.
2) Incubate 5 minutes at room temperature and centrifuge for 15 s at >15.000×g.
3) Continue with your usual protocol (washing steps) of your isolation procedure.

Every activated carboxylic acide, like anhydrides, halides, are also possible to react as reactive compound. Other catalysts like tetrabutylammonium fluoride, tetrabutylammonium bromide, aminopyridine or 4-dimethylaminopyridine (generally: amino-functional compound) may also be used. Suitable modification and catalysts are known in the prior art in order to modify the polynucleotide comprising ribose rings with the modifications described above.

### EXAMPLE 3

According to the literature, modified RNA changes its secondary structure thereby making it an inappropriate substrate for RNA-dependent enzymes like a reverse transcriptase commonly used to reverse transcribe RNA into complementary DNA (cDNA) which is subsequently used in polymerase-chain reaction to detect and potentially quantitate specific RNA molecules contained in a sample. Modified RNA as being described in the example below, however, needs to be re-modified via complex and tedious steps in order to make it a suitable substrate for such reactions. In contrast, a modified polymerase or temperatures/buffer conditions being able to work with such structural changes or reverting such changes in secondary structure, respectively, will make modified RNA directly a substrate for a polymerase e.g. in PCR applications resulting in a much more convenient and streamlined procedure.

2 μg of RNA isolated with a RNeasy Mini Kit (Qiagen, Hilden) according to the standard protocol was modified according to the STABMRT Mini Kit protocol (RNAworks, Montpellier).

Modified RNA was pretreated to change structure:

a) no pretreatment
b) 15 mins 95° C.; cool down at room temperature (slowly)
c) 15 mins 95° C., cool down at −20° C. (rapid)

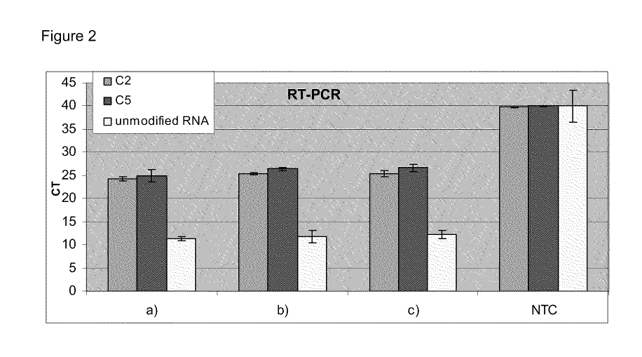

50 ng each were used in a RT-qPCR for GAPDH as template (QuantiTect SYBR Green RT-PCR Kit; Qiagen, Hilden). FIG. 2 shows the results. The abbreviations have the following meanings:

Error bar: range

NTC: non template control

Surprisingly, with modified RNA, which is not thought to be a target for the reverse transcriptase due to the changed secondary structure, a clear signal is obtained. Different pre-treatment procedures show no difference in the performance of the reaction. This means that the RT is able to recognize the modified RNA with the changed secondary structure as a template. Even if the performance is currently worse compared to unmodified RNA this clearly demonstrates that with a new polymerase optimized to the “new” RNA-structure the PCR could be done with the same performance than the current enzyme to the “classical” RNA-structure. The same principle shall hold true for other polymerases as well as RT enzymes.

1. A method of performing a detection and/or polymerisation reaction, comprising:
using a modified polynucleotide comprising ribose rings, wherein at least part of said ribose rings comprise a modification at the 2′-OH position, wherein said modification enables immobilisation of said modified polynucleotide to a support
immobilising said modified polynucleotide to a support
performing a detection and/or polymerisation reaction using the polynucleotide as a template and/or target for detection.

2. The method according to claim 1, wherein said ribose rings are modified with at least two different modifications.

3. The method according to claim 1, wherein the percentage of modification is less than 75%.

4. The method according to claim 1, wherein said modification is covalent and/or reversible.

5. The method according to claim 1, wherein said modification is covalent and is selected from the group consisting of:
modifications altering the overall charge of said polynucleotide,
modifications providing an affinity tag to said polynucleotide,
modifications altering the hydrophobicity of said polynucleotide,
modifications altering the affinity to thiophillic matrices, and
reversible modifications introducing a light-sensitive side chain.

6. The method according to claim 1, wherein said modification comprises a substituent OR or OR′, wherein R is selected from the group consisting of alkyl, alkenyl, alkynyl, haloalkyl, aminoalkyl, haloalkoxyalkyl, aminoalkoxyalkyl, aryl, alkylaryl, arylalkyl, arylalkenyl, alkanoyl, alkenoyl, haloalkanoyl, dihaloalkanoyl, trihaloalkanoyl, haloformylalkanoyl, aminoalkanoyl, arylalkanoyl, arylalkenoyl, alkoxyalkanoyl, aryloxyalkanoyl, alkylarylalkanoyl, azidoalkanoyl, carboxyalkanoyl, carboxyalkenoyl, carboxyalkynoyl, haloarylalkanoyl, aminoarylalkanoyl, alkylaminoarylalkanoyl, haloalkenoyl, haloalkynoyl, alkylsilanyl, trialkylsilanyl alkoxycarbonyl, alkylthioalkoxyalkoxycarbonyl, alkenyloxycarbonyl, alkoxyalkoxyalkyl, alkoxyalkyl, alkylthioalkyl, alkylsulfonyl, and diarylphosphone, and wherein the aforementioned substituents may be optionally substituted; or a substituent R′, wherein R′ is selected from alkyl, alkenyl, alkynyl, haloalkyl, aminoalkyl, halo, amino, alkylamino, aryl, alkylaryl, and/or arylalkyl, and wherein the aforementioned substituents may be optionally substituted.

7. The method according to claim 6, wherein R and/or R′ is selected from
methyl, ethyl, vinyl, allyl, ethynyl, 2-chloroethyl, 2-aminoethyl, ethyloxyethyl, methoxymethyl, methylthiomethyl, methoxyethoxymethyl, (2-chloroethyl)oxyethyl, (2-aminoethyl) oxyethyl, phenyl, 4-methylphenyl, benzyl, cinnamyl, formyl, acetyl, propanoyl, butanoyl, pentanoyl, hexanoyl, heptanoyl, octanoyl, nonanoyl, pivaloyl, isobutanoyl, isopentanoyl, carboxyacetyl, chloroformylnonanoyl, 3-carboxypropanoyl, 4-aminobutanoyl, 4-chlorobutanoyl chloroacetyl, dichloroacetyl, trifluoroacetyl, trichloroacetyl, 3-azidopropanoyl, 4-azidobutyryl acryloyl, propioloyl, crotonoyl, benzoyl, diphenylacetyl, phenoxyacetyl, methoxyacetyl, methoxycarbonyl, 2-(methylthiomethoxy)ethoxycarbonyl, vinyloxycarbonyl, 4-methylbenzoyl, 4-chlorobenzoyl, 2-methylaminobenzoyl, 2-aminobenzoyl, 4-aminobenzoyl, 4-nitrobenzoyl, cinnamoyl, silanyl, trimethylsilanyl, triethylsilanyl, tripropylsilanyl, triisopropylsilanyl, t-butyldimethylsilanyl, 2-chlorophenyl (4-nitrophenyl)phosphono, and/or methylsulfonyl; and R′ is selected from methyl, ethyl, vinyl, allyl, ethynyl, t-butyl, 2-chloroethyl, 2-aminoethyl, ethyloxyethyl, phenyl, benzyl, fluoro, chloro, bromo, iodo, and/or amino.

8. The method according to claim 1, wherein said modification is selected from the group consisting of
polyacrylic acid or polyaspartatic acid
polyethylenimine, polyvinylamine or polylysine
C1 to C20 or C6 to C20, carbon chains or perfluorinated carbon chains
Biotin or poly-histidine
siloxane with a formulation of “—O—SiR₃”, where R can be an organic moiety with 1 to 6 carbons, a phenyl residue or a “—O—SiR₃” group with R=aliphatic group CₙH₂ₙ₊₁ with n=1-8 or aromatic group, and
modifications comprising a label.

9. The method according to claim 1, wherein said support is selected from the group consisting of nucleic acid binding matrices, membranes, particles, and laboratory equipment.

10. A method for isolating, purifying and/or preparing a target nucleic acid from a mixture of at least two different types of nucleic acids, wherein one of said nucleic acids is a polynucleotide comprising ribose rings, said method comprising the following steps:
contacting said polynucleotide comprising ribose rings with a reactant capable of modifying the 2′-OH position of the ribose rings of said polynucleotide
reacting said polynucleotide with said reactant to produce a modified polynucleotide wherein at least part of said ribose rings comprise a modification at the 2′-OH position, wherein said modification changes the chemical and/or physical properties of said polynucleotide, and
isolating the target nucleic acid.

11. The method according to claim 10, wherein said modified polynucleic acid is immobilised via said modification to a support for isolation, purification and/or preparation.

12. The method according to claim 11, wherein said support is a nucleic acid binding matrix.

13. The method according to claim 10, wherein the modification of the polynucleotide comprising ribose rings is performed by at least one of the following steps
adding a modifying reagent during lysis of the sample comprising the target nucleic acid
adding a modifying reagent when the polynucleotide comprising ribose rings is contacted with a nucleic acid binding matrix
adding a modifying reagent while the polynucleotide comprising ribose ring is adsorbed to a nucleic acid binding matrix.

14. The method according to claim 10, wherein said modification comprises said ribose rings being modified with at least two different modifications.

15. A method for selecting a polymerization agent having the ability to efficiently utilize a polynucleotide comprising ribose rings wherein at least part of said ribose rings comprise a modification at the 2′-OH position, wherein said modification changes the chemical and/or physical properties of said polynucleotide, said method comprising the steps of:
mutagenising said polymerisation agent
incubating the mutagenised polymerisation agent together with said modified polynucleotide, and
detecting the efficiency of said polymerisation reaction.

16. The method according to claim 15, wherein at least 25% of the ribose rings are modified.

17. The method of claim 15, wherein the method comprises employing a reaction buffer comprising a salt, dNTP and at least one oligonucleotides.

18. The method of claim 15, wherein said method is at least part of an isothermal amplification reaction and/or a polymerase chain reaction.

19. A method of claim 8, wherein said label is selected from the group consisting of a fluorescent label, a radioactive label, an enzyme, a ligand and an affinant for a label.
